(19) United States
(12) Patent Application Publication
(10) Pub. No.: US 2009/0324493 A1
(43) Pub. Date: Dec. 31, 2009

(54) METHOD FOR PREPARING A SALINE SOLUTION COMPRISING [13N] NH4 + AND USE OF A DEVICE

(75) Inventors: Paul Burke, Cambridgeshire (GB); Hannu Sipilä, Turku (FI); John Charles Clark, Buckinghamshire (GB)

Correspondence Address: JAMES C. LYDON, 100 DAINGERFIELD ROAD, SUITE 100, ALEXANDRIA, VA, 22314, US

(73) Assignee: HIDEX OY, Turky (FI)

(21) Appl. No.: 11/919,425

(22) PCT Filed: Apr. 25, 2006
(86) PCT No.: PCT/FI2006/000130

§ 371 Date: Sep. 3, 2009

(30) Foreign Application Priority Data
Apr. 26, 2005 (FI) 20050432

(51) Int. Cl. A61K 51/02

(52) U.S. Cl. 424/1.61

(57) ABSTRACT

The invention relates to a method for preparing a saline solution comprising [13N]NH4+. The method comprises following steps: preparing [13N]NH3 gas, transporting [13N]NH3 gas to a diffusion chamber, to a first side of a semipermeable membrane, transporting sterile saline solution to the diffusion chamber, to a second side of a semipermeable membrane, allowing the mixing of [13N]NH3 gas that has penetrated the semipermeable membrane with the saline solution on the second side of the semipermeable membrane, whereby saline solution comprising [13N]NH4+ is obtained, and directing the saline solution comprising [13N]NH4+ out from the diffusion chamber. The invention relates also to use of a device for preparing a radiolabelled saline solution for preparation of a saline solution comprising [13N]NH4+.

The present invention relates to a method for preparing a saline solution comprising [¹³N]NH₄⁺ and use of a device defined in the preambles of the independent claims presented hereafter.

Positron emission tomography (PET) is a quantitative, functional isotope imaging method used, for instance, for the examination of blood flow in the human heart, brain or skeletal muscles. Ammonia labelled with the short-lived nitrogen-13-isotope, (half-life 9.965 minutes, 100% β⁺, decay) can be used as the radioactive tracer and it is the most important of [¹³N]radiolabelled compounds used in PET imagining studies. It is highly diffusible tracer, which is effectively extracted into imaging tissues. The [¹³N]ammonia is usually administered as a physiological sodium chloride solution in water.

[¹³N]ammonia is usually prepared in a cyclotron by bombardment of a water target with beam of protons inducing ¹⁶O(p, α)¹³N nuclear reaction. [¹³N]ammonia can be prepared either by direct in-target ammonia formation by irradiation of a water-ethanol mixture or by producing a mixture of nitrous oxides from a pure water target which nitrous oxides subsequently are further processed chemically to yield ammonia. In both cases [¹³N]ammonia is obtained in gas form and collected in sterile saline solution, e.g. by bubbling the [¹³N]ammonia gas through the saline solution. The labelled saline solution is then removed batchwise from the collection apparatus e.g. by a syringe or the like, and after sterile filtration/quality control analysis medical personnel takes the labelled solution to the patient for administration. This procedure exposes the medical personnel for possible radioactive exposure, and requires special care and attention

It has not been generally believed that [¹³N]ammonia is transportable in gas form over relatively long distances, because it has been thought that the trace amounts of [¹³N]ammonia adhere to the inner walls of used conduits.

It is known to produce radioactive water labelled with ¹⁵O by using an apparatus described in U.S. Pat. No. 6,858,187. The device comprises a reaction chamber for formation of radioactive water vapour, a diffusion chamber which allows the radioactive water vapour to penetrate, but which prevents the penetration of carrier gases, and tubes and valves for directing a sterile saline solution to the diffusion chamber, for directing the saline solution containing radioactive water out from the diffusion chamber and further to a patient, or to a decay coil, and a radioactivity measuring instrument. The diffusion chamber, the tubes, the valves, and the radioactivity measuring instrument are mounted in the same frame, whereby they form a separate unit, which as one entity can be detached from the lead shield surrounding the device.

The object of the present invention is to minimise or even to totally eliminate the above-mentioned problems.

The object is thus to provide a simple method for preparation a saline solution comprising [¹³N]NH₄⁺ that would result in a sterile pyrogen free [¹³N]NH₄⁺ labelled saline solution.

Another object of the present invention is to provide a method that allows simple transportation of the [¹³N]NH₄⁺ labelled saline solution to the patient with minimum risk of radioactive dose to the personnel.

In order to achieve the above-mentioned objects the present invention is characterised in what is defined in the characterising parts of the independent claims presented hereafter.

Typical method according to the present invention for preparing a saline solution comprising [¹³N]NH₄⁺ comprises following steps:
preparing [¹³N]NH₃ gas,
   characterised in
transporting [¹³N]NH₃ gas to a diffusion chamber, to a first side of a semipermeable membrane,
transporting sterile saline solution to the diffusion chamber, to a second side of a semipermeable membrane,
allowing the mixing of [¹³N]NH₃ gas that has penetrated the semipermeable membrane with the saline solution on the second side of the semipermeable membrane, whereby saline solution comprising [¹³N]NH₄⁺ is obtained, and
directing the saline solution comprising [¹³N]NH₄⁺ out from the diffusion chamber.

According to a typical embodiment of the present invention a device for preparing a radiolabelled saline solution, which device comprises a diffusion chamber comprising at least one semipermeable membrane, tubes and valves for directing a sterile saline solution to the diffusion chamber to a first side of the membrane, tubes and valves for directing a radiolabelled gas to the diffusion chamber to a second side of the membrane, is used for preparation of a saline solution comprising [¹³N]NH₄⁺.

Now it has been surprisingly found out that a saline solution comprising [¹³N]NH₄⁺ can be produced by transporting the [¹³N]NH₃ gas to a diffusion chamber, where the gas is allowed to penetrate through a semipermeable membrane to a sterile saline solution. By using the method of the present invention a saline solution comprising [¹³N]NH₄⁺ can be obtained in a simple and effective manner. Quite surprisingly it has also been found out that [¹³N]NH₃ gas can be transported relatively long distances, without significant loss of radioactivity.

According to one preferred embodiment of the present invention [¹³N]NH₃ gas is transported to a diffusion chamber, to a first side of a semipermeable hydrophobic membrane. The diffusion chamber can comprise a second semipermeable membrane, which is hydrophilic. Sterile saline solution is transported to the diffusion chamber, to a first side of this second semipermeable membrane. The [¹³N]NH₃ gas that has penetrated the hydrophobic membrane is allowed to mix with the saline solution that has penetrated the hydrophilic membrane in a space between the membranes. Thus saline solution comprising [¹³N]NH₄⁺ is obtained.

When the radiolabelled saline solution is prepared in this manner, the resulting solution is sterile and pyrogen free.

According to one embodiment of the invention [¹³N]NH₃ gas is transported from the apparatus in which it is produced to a diffusion chamber via a tube having a length of at least 5 metres, typically at least 10 metres, usually 15 metres, preferably 20 metres, more preferably 30 metres. The length of the tubing is, however, usually shorter than 70 metres, usually shorter than 60 metres, preferably shorter than 50 metres. The tubes are preferably of stainless steel, teflon® or the like.

According to one preferred embodiment of the invention the [¹³N]NH₃ gas is dried before it is transported to the diffusion chamber. Drying can be achieved by using a NaOH trap or the like. Thus the dryness of the [¹³N]NH₃ gas can be secured, as well as the optimal working of the diffusion chamber. Without drying minute amounts of water vapour may be transported to the diffusion chamber. Wet [¹³N]NH₃ gas is also easily attached to the walls of the tubes and piping used for transporting the gas to the diffusion chamber.

The invention is described in more detail with the aid of the following FIGURE, in which

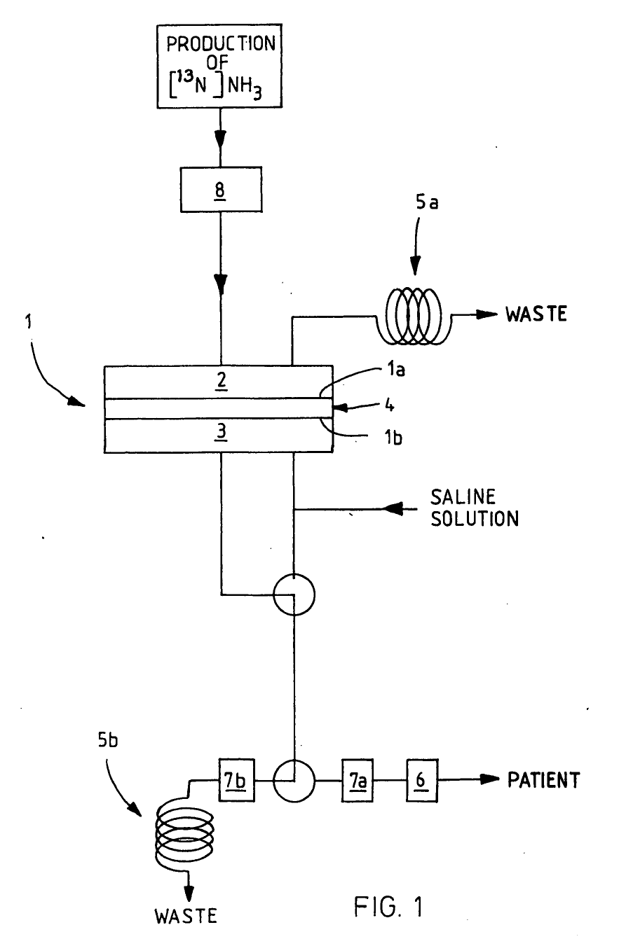

FIG. 1 shows a preparation of saline solution comprising [¹³N]NH₄⁺ according to one embodiment of the invention as a schematic flow diagram.

In FIG. 1 the radioactive [¹³N]NH₃ gas is prepared in a manner known per se, e.g. by irradiation of [¹⁶O] water in a standard water target via the ¹⁶O(p,α)¹³N nuclear reaction. Irradiated water is flushed out from the target chamber into a reduction chamber, where the gaseous [¹³N] ammonia is released.

The apparatus for producing [¹³N]NH₃ gas is connected to the diffusion chamber 1 via a tube, in which case the distance between the apparatus for producing [¹³N]NH₃ gas and the diffusion chamber can be very long, for example 20 or 30 meters. A gas dryer 8, e.g. NaOH trap or dryer, is located before diffusion chamber 1, for drying the gas before it enters the diffusion chamber.

The diffusion chamber 1 has two membranes 1a and 1b, which separate the gas phase 2 from the liquid phase 3. The upper membrane 1a which is in contact with the gas phase 2 is a hydrophobic membrane, for instance of the type Millipore GVHP, and the lower membrane 1b which is in contact with the liquid phase 3 is a hydrophilic membrane, for instance of the type Millipore GSTF. Sterile sodium chloride solution is supplied by an infusion pump, not shown in the FIGURE, to the solution side 3 of the diffusion chamber. The [¹³N]NH₃ gas which has penetrated the membrane 1a condenses and mixes with the sterile saline solution in the space 4 between the membranes 1a and 1b of the diffusion chamber 1. The hydrophilic membrane 1b effectively prevents the penetration of carrier gases, and they are discharged as waste gases to a decay coil 5a. The radioactive labelled saline solution is supplied through a sterile filter 6 to a patient or the decay coil 5b. The radioactivity of the solution, which is transported to the patient can be measured with a radioactivity measuring instrument 7a and the radioactivity of the solution, which is transported to the decay coil, can be measured with a radioactivity measuring instrument 7b.

### EXAMPLE

[¹³N] was produced by irradiation of 1 ml [¹⁶O] water (Millipore Milli-Q) in a standard GE silver water target with 5 μA of 16 MeV protons GE PET trace via the ¹⁶O(p,α)¹³N reaction. After 30 s irradiation about 200 MBq of [¹³N] was produced. Irradiated water was flushed out from the target chamber with helium gas, 250 ml/min, into a reaction vessel containing 1 g of DeVarda's alloy and 1 mg (1 ml) of sodium nitrite as a carrier. 3 ml of 10 M NaOH was added to convert the [¹³N] nitrate/nitrite to [¹³N]ammonia. The gaseous output of the reaction vessel was connected through a NaOH water trap to the gas inlet of the diffusion chamber similar to the type used for radio water preparation. The liquid compartment of the diffusion chamber was filled with saline.

After 1 minute the reaction vessel and the diffusion chamber were flushed with helium for 4 minutes. Helium flow was stopped and 10 ml of saline was flushed through the liquid compartment of the diffusion chamber into a collection vessel.

The yield of ammonia was 71% using 20 m long 3 mm OD polypropylene tubing between the reaction vessel and the diffusion chamber. It can be concluded that [¹³N]ammonia can be trapped with the same type of diffusion chamber as is used for radio water production and described in U.S. Pat. No. 6,8585,187. Similarly, it can be concluded that [¹³N] ammonia is transportable in the gas phase. This enables the construction of a bed-side [¹³N]ammonia infuser similar to that described in U.S. Pat. No. 6,8585,187 for radio water labeled with ¹⁶O.

It will be appreciated that the essence of the present invention can be incorporated in the form of a variety of embodiments, only a few of which are disclosed herein. It will be apparent for the specialist in the field that other embodiments exist and do not depart from the spirit of the invention. Thus, the described embodiments are illustrative and should not be construed as restrictive.

1. A method for preparing a saline solution comprising [¹³N]NH₄⁺, the method comprising following steps:
preparing [¹³N]NH₃ gas,
characterised in
transporting [¹³N]NH₃ gas to a diffusion chamber, to a first side of a semipermeable membrane,
transporting sterile saline solution to the diffusion chamber, to a second side of a semipermeable membrane,
allowing the mixing of [¹³N]NH₃ gas that has penetrated the semipermeable membrane with the saline solution on the second side of the semipermeable membrane, whereby saline solution comprising [¹³N]NH₄⁺ is obtained, and
directing the saline solution comprising [¹³N]NH₄⁺ out from the diffusion chamber.

2. Method according to claim 1, characterised in
transporting [¹³N]NH₃ gas to a diffusion chamber, to a first side of a semipermeable hydrophobic membrane,
transporting sterile saline solution to the diffusion chamber, to a first side of a second semipermeable hydrophilic membrane, and
allowing the mixing of [¹³N]NH₃ gas that has penetrated the hydrophobic membrane with the saline solution that has penetrated the hydrophilic membrane in a space between the membranes, whereby saline solution comprising [¹³N]NH₄⁺ is obtained.

3. Method according to claim 1, characterised in that the [¹³N]NH₃ gas is transported to a diffusion chamber via a tube having a length of at least 5 metres.

4. Method according to claim 1, characterised in drying the [¹³N]NH₃ gas before it is transported to the diffusion chamber.

5. Use of a device for preparing a radiolabelled saline solution, the device comprising
a diffusion chamber comprising at least one semipermeable membrane
tubes and valves for directing a sterile saline solution to the diffusion chamber to a first side of the membrane
tubes and valves for directing a radiolabelled gas to the diffusion chamber to a second side of the membrane,
for preparation of a saline solution comprising [¹³N]NH₄⁺.

6. Use according to claim 5, characterised in that the device comprises at least two semipermeable membranes of which one is hydrophilic and one hydrophobic, whereby a space is formed between the membranes for mixing the saline solution and the radiolabelled gas.
